(19) United States
(12) Patent Application Publication
(10) Pub. No.: US 2014/0038228 A1
(43) Pub. Date: Feb. 6, 2014

(54) REMOVABLE LAYER AND METHOD OF USE

(71) Applicants: Neil Percy, St.Paul, MN (US); Gregory W. Sitton, Minneapolis, MN (US); Tonya D. Bonilla, Lake Elmo, MN (US)

(72) Inventors: Neil Percy, St.Paul, MN (US); Gregory W. Sitton, Minneapolis, MN (US); Tonya D. Bonilla, Lake Elmo, MN (US)

(73) Assignee: 3M INNOVATIVE PROPERTIES COMPANY, St. Paul, MN (US)

(21) Appl. No.: 14/110,949

(22) PCT Filed: Apr. 19, 2012
(86) PCT No.: PCT/US12/34152

§ 371 Date: Oct. 10, 2013

Related U.S. Application Data
(60) Provisional application No. 61/478,237, filed on Apr. 22, 2011.

(51) Int. Cl. C12Q 1/24

(52) U.S. Cl. CPC C12Q 1/24
USPC 435/30; 435/309.2

(57) ABSTRACT

A method of processing a sample for analysis is disclosed. The method includes providing a first sample-transfer article with a sample disposed therein, a container (1110), a pierceable cap (1120), and a pierceable layer (1500). The method further includes operably coupling the cap (1120) and the container (1110), disposing the pierceable layer (1500) adjacent the cap (1120), urging the first sample-transfer article through the layer (1500) and the cap (1120), transferring a portion of the sample into the container (1110), and separating the layer (1500) from the cap (1120). The method further can comprise providing a second sample transfer article having a second sample delivery end and urging the second sample-transfer article through the cap. A kit comprising a container, a cap, a pierceable layer; and means for coupling the pierceable layer to the cap and/or to the container is also provided.

### CROSS REFERENCE TO RELATED APPLICATIONS

This application claims the benefit of U.S. Provisional Patent Application No. 61/478,237, filed Apr. 22, 2011, which is incorporated herein by reference in its entirety.

### BACKGROUND

Applications of nucleic acid, protein, or antigen testing are broad. The majority of current commercial testing relates to infectious diseases including Chlamydia, gonorrhea, hepatitis and human immunodeficiency virus (HIV) viral load; genetic diseases including cystic fibrosis; coagulation and hematology factors including hemochromatosis; and cancer. Food and beverages are also tested for the presence or absence of potentially-pathogenic microorganisms. The majority of testing currently occurs in centralized laboratories using non-portable and operationally complex instruments. Presently, tests generally require highly skilled individuals to perform the assays.

Nucleic acids found in cells can be deoxyribonucleic acid or ribonucleic acid and can be genomic DNA, extrachromosomal DNA (e.g. plasmids and episomes), mitochondrial DNA, messenger RNA and transfer RNA. Nucleic acids can also be foreign to the host and contaminate a cell as an infectious agent, e.g. bacteria, viruses, fungi or single celled organisms and infecting multicellular organisms (parasites).

The detection of non-nucleic acid materials (e.g., proteins, polysaccharides) is also used to detect the presence or absence of a microorganism in a sample or in the environment. Immunochromatography and ELISA tests can be used to detect the presence of such molecules.

Methods of extracting analytes (e.g., nucleic acids, proteins) from cells are known to those skilled in the art. The specific method of nucleic acid, protein, or polysaccharide extraction may be dependent on the type of molecule to be isolated, the type of cell, and the specific application used to analyze the molecule. Many methods of isolating DNA are known to those skilled in the art, see for example the general reference Sambrook and Russell, 2001, “Molecular Cloning: A Laboratory Manual”.

Methods of releasing nucleic acids from cells are well known to those skilled in the art. Typically, cell disruption is performed using mechanical means (e.g., sonic vibration, heat) and/or chemical means (e.g., strong base, detergent, chaotropic agents).

There is a need for efficient methods to test for the presence of pathogenic microorganisms in a sample.

### SUMMARY

In general, the invention is directed to a method of evaluating the effectiveness of a cleaning and/or disinfecting process.

In one aspect, the present disclosure provides a method of processing a sample for analysis. The method can comprise providing a first sample transfer article, a container, a cap, and a pierceable layer. The first sample transfer article can have a first sample delivery end and a sample reservoir having a sample disposed therein. The container can comprise an opening and an inner chamber. The cap can be shaped and proportioned to couple with the container and seal the opening. The cap can have an inner surface and an outer surface. The method further can comprise operably coupling the cap with the container, disposing the layer adjacent a portion of the outer surface, urging the first sample delivery end through the layer and the cap, transferring a portion of the sample into the container, and separating the layer from the outer surface.

In any of the above embodiments, providing a cap further can comprise providing a cap that includes an elastically-deformable slit extending from the outer surface to the inner surface. In these embodiments, urging the first sample delivery end through the cap further can comprise urging the first sample delivery end through the cap via the slit.

In any of the above embodiments, the method further can comprise providing a second sample transfer article having a second sample delivery end and urging the second sample delivery end through the cap. In some embodiments, urging the second sample delivery end through the cap further can comprise urging the second sample delivery end through the cap. In some embodiments, urging the second sample delivery end through the cap further comprises urging the second sample delivery end through the slit.

In any of the above embodiments, disposing the layer adjacent the outer surface further can comprise coupling the layer to the outer surface.

In any of the above embodiments providing a container and a cap further can comprise providing the container with the cap operably coupled there to. In any of the above embodiments, providing a cap and a pierceable layer further can comprise providing the cap with the layer coupled to the outer surface. In some embodiments, coupling the layer to the outer surface further comprise detachably coupling the layer to the outer surface.

In any of the above embodiments, the pierceable layer can comprise a water-absorbent nonwoven material, wherein urging the sample delivery end through the layer further can comprise absorbing into the layer at least a portion of sample adhered to the outside of the sample transfer article.

In another aspect, the present disclosure provides an article. The article can comprise a container, a cap, and a pierceable layer. The container can comprise an opening and an inner chamber. The cap can be shaped and proportioned to couple with the container and seal the opening. The cap can have an inner surface, an outer surface, and a pierceable region to permit the passage of the sample delivery end through the cap and into the inner chamber. The cap can be operably coupled with the container and the layer can be coupled to a portion of the outer surface. In some embodiments of the article, the layer can be detachably coupled to the outer surface.

In any of the above embodiments of the article, the cap further can comprises a pierceable region to permit the passage of a sample delivery end of a sample transfer article through the cap and into the inner chamber. In some embodiments, the pierceable region further can comprise an elastically-deformable slit extending from the outer surface to the inner surface. In any of the above embodiments, the article further can comprise a cell lysis agent disposed in the inner chamber. In any of the above embodiments, the article further can comprise an aqueous liquid disposed in the inner chamber.

In yet another aspect, the present disclosure provides a kit. The kit can comprise a container, a cap, a pierceable layer, and a means for coupling the pierceable layer to the cap and/or to the container. The container can comprise an opening and an inner chamber. The cap can be shaped and proportioned to couple with the container and seal the opening. The cap can have an inner surface, an outer surface, and a pierceable region to permit the passage of a sample delivery end of a sample transfer article through the cap.

In any embodiment of the kit, the pierceable layer can comprise a film or a nonwoven material. In any embodiment of the kit, the pierceable layer can comprise a water-absorbent nonwoven material. In any embodiment of the kit, the pierceable layer can comprise a perforated polyethylene film and/or a porous paper tape. In any embodiment, the kit further can comprise a sample transfer article. In any embodiment, the kit further can comprise a reagent that facilitates cell lysis. In some embodiments, the reagent that facilitates cell lysis can be disposed in the container. In any embodiment of the kit, the reagent that facilitates cell lysis can be selected from the group consisting of a detergent, and antibiotic, and a polypeptide. In any embodiment of the kit, providing a cap further can comprise providing an article comprising a plurality of connected, spaced-apart caps, wherein each spaced-apart cap is shaped and proportioned to seal the opening of one of a plurality of containers. In any embodiment of the kit, the container further can comprise a liquid disposed in the inner chamber. In any embodiment, the kit further can comprise a detection reagent or a detection device.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a container can be interpreted to mean “one or more” containers.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a block diagram of one embodiment of a method for detecting a target microorganism according to the present disclosure.

FIG. 2 is a side view of one embodiment of a sample-transfer article according to the present disclosure.

FIG. 3 is an exploded side view of one embodiment of a container with a cap, according to the present disclosure.

FIG. 4A is a plan view of the outer surface of the cap of FIG. 3.

FIG. 4B is a plan view of the inner surface of the cap of FIG. 3.

FIG. 5A is a side view, partially in section, of one embodiment of a pierceable layer disposed adjacent to the cap of FIGS. 4A-B, which is operably coupled with the container of FIG. 3.

FIG. 5B is a side view, partially in section, of the sample-transfer article of FIG. 5A inserted through the pierceable layer and cap of FIG. 5A.

FIG. 5C is a side view, partially in section, of the sample transfer article, container, cap, and pierceable layer of FIG. 5B as the sample transfer article is delivering a liquid sample into the container.

FIG. 6 is a cross-sectional view of the pierceable layer of FIG. 5A.

FIG. 7 is an exploded side view of one embodiment of an assembly for processing a plurality of samples

FIG. 8 is a side view of the assembly of FIG. 7, showing the removal of the pierceable layer strip.

### DETAILED DESCRIPTION

The present disclosure generally relates to an article and a method for processing a sample for analysis. In particular, the disclosure relates to the transfer of all or a portion of a liquid sample to be tested into a container (e.g., a tube with a pierceable cap) in which the sample is processed. Positioned adjacent the pierceable cap is a pierceable layer. The sample may be any liquid-containing sample as described herein. Typically, the sample is transferred into the container by inserting a sample transfer article (e.g., a pipette or a pipette tip attached to a pipettor) through the pierceable layer and pierceable cap and depositing the sample into the container. The method of the present disclosure is particularly advantageous when the liquid sample comprises suspended solids and/or liquids (e.g., viscous liquids) that have a tendency for droplets of the sample to adhere to the outer surface of the sample transfer article. Thus, as the sample-transfer article passes through the pierceable layer, residual liquid droplets and solids adhered to the outside of the sample-transfer article are retained by the pierceable layer and, thereby, are not transferred onto the pierceable cap. Advantageously, the inventive method provides a way to introduce a liquid sample into a container without removing a cap from the container and without leaving a residue on the cap that could lead to later contamination of the sample and/or cross-contamination of other samples proximate the container. A further advantage of the method is present when processing a plurality of samples. After depositing a sample into each container, the pierceable layer provides a visible indication (e.g., a hole in the layer) of the last container (e.g., in a row) that received a sample.

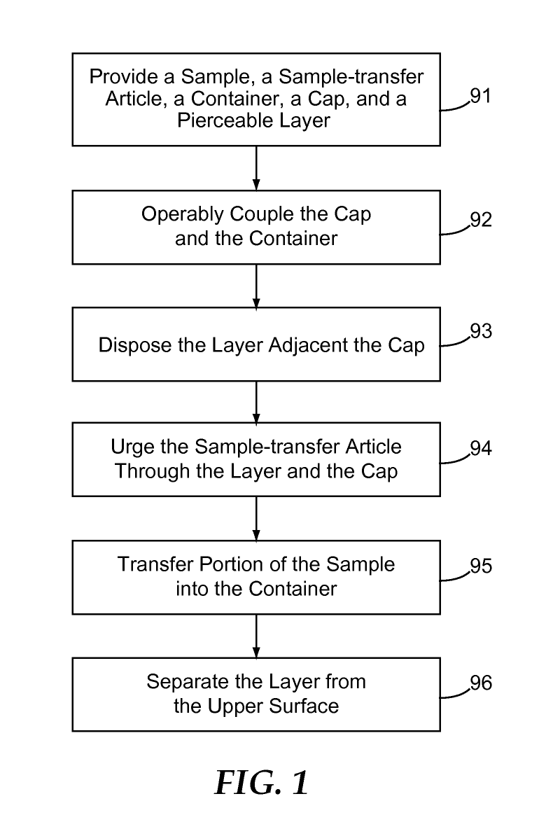

FIG. 1 shows a block diagram of one embodiment of a method of processing a sample according to the present disclosure. The method includes the step 91 of providing a sample, a sample-transfer article, a container, a cap, and a pierceable layer.

Providing a sample to be tested may comprise providing a sample that is suspected of containing a target microorganism. The sample can be any sample that may include a target microorganism as defined herein. Nonlimiting examples of suitable samples include environmental samples (e.g., surface swabs/sponges, soil, sediments, fomites), food (e.g., raw materials, in-process samples, and finished-product samples), beverages, clinical/veterinary samples (e.g., blood, serum, plasma, urine, sputum, tissue, mucous, feces, wound exudate, pus, cerebrospinal fluid), and water (e.g., surface water, potable water, process water).

In some embodiments, the presence or absence of a target microorganism can be analyzed in a test sample that is derived from a variety of food, beverage, or food- or beverage-processing environmental sources. Non-limiting examples of food sources include raw or processed meat, raw or processed fruits or vegetables, non-fluid dairy products (e.g., cheese, butter, and ice cream), nuts, spices, ingredients, and syrups. Non-limiting examples of beverage sources include potable water, fruit or vegetable juices, milk, and fermented beverages. Pasteurized food or beverages may also be suitable sources. Non-limiting examples of food- or beverage-processing environmental samples include food-handling surface samples (e.g., conveyor belts, blades, cutting surfaces, mixing equipment surfaces, filters, storage containers), room samples (e.g., walls, floors, drains, ventilation equipment), and cleaning equipment (e.g., hoses, cleaning tools).

In some embodiments, the presence or absence of a target microorganism can be analyzed in a sample that is derived from a variety of human or animal sources, such as a physiological fluid, e.g., blood, saliva, ocular lens fluid, synovial fluid, cerebral spinal fluid, pus, sweat, exudate, urine, mucus, lactation milk, or the like. Further, the test sample may be derived from a body site, e.g., wound, skin, nares, scalp, nails, etc.

Samples of particular interest from human or animal sources include mucus-containing samples, such as nasal samples (from, e.g., anterial nares, nasopharyngeal cavity, nasal cavities, anterior nasal vestibule, etc.), as well as samples from the outer ear, middle ear, mouth, rectum, vagina, or other similar tissue. Examples of specific musosal tissues include buccal, gingival, nasal, ocular, tracheal, bronchial, gastrointestinal, rectal, urethral, ureteral, vaginal, cervical, and uterine mucosal membranes.

Besides physiological fluids, other test samples may include other liquids as well as solid(s) dissolved or suspended in a liquid medium. Samples of interest may be obtained from process streams, water, soil, plants or other vegetation, air, surfaces (e.g., contaminated surfaces), and the like. Samples can also include cultured cells. Samples can also include samples on or in a device comprising cells, spores, or enzymes (e.g., a biological indicator device).

Suitable samples for methods of the present disclosure can include certain solid samples. Solid samples may be disintegrated (e.g., by blending, sonication, homogenization) and may be suspended in a liquid (e.g., water, buffer, broth). In some embodiments, a sample-collection device (e.g., a swab, a sponge) containing sample material may be used in the method. Alternatively, the sample material may be eluted (e.g., rinsed, scraped, expressed) from the sample-collection device before using the sample material in the method. In some embodiments, liquid or solid samples may be diluted in a liquid (e.g., water, buffer, broth).

The sample may comprise an indicator microorganism, as described herein. The indicator microorganism can be indicative of contamination (e.g., fecal contamination), infection (e.g., infection with a pathogenic microorganism), or an indicator of general sanitation (e.g., any aerobic microorganism). The indicator microorganism further can be a target microorganism.

Microorganisms of particular interest, which may be of interest as an indicator organism or a target microorganism, include prokaryotic and eukaryotic organisms, particularly Gram positive bacteria, Gram negative bacteria, fungi, mycoplasma, and yeast. Particularly relevant organisms include members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp. as well as herpes virus, Aspergillus spp., Fusarium spp., and Candida spp. Particularly virulent organisms include Staphylococcus aureus (including resistant strains such as Methicillin Resistant Staphylococcus aureus (MRSA)), S. epidermidis, Streptococcus pneumoniae, S. agalactiae, S. pyogenes, Enterococcus faecalis, Vancomycin Resistant Enterococcus (VRE), Vancomycin Resistant Staphylococcus aureus (VRSA), Vancomycin Intermediate-resistant Staphylococcus aureus (VISA), Bacillus anthracia, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Cronobacter sakazakii, E. coli O157 and multiple drug resistant Gram negative rods (MDR).

Gram positive and Gram negative bacteria are of particular interest. Of particular interest are Gram positive bacteria, such as Listeria monocytogeness. Also, of particular interest are antibiotic resistant microbes including MRSA, VRSA, VISA, VRE, and MDR microbes.

In some embodiments, the sample can be mixed, suspended, and/or diluted in a liquid suspending medium. The liquid suspending medium can be an aqueous liquid such as water or a buffer solution (e.g., phosphate-buffered saline), for example. Samples comprising solid material can be suspended, and optionally homogenized, in the liquid suspending medium.

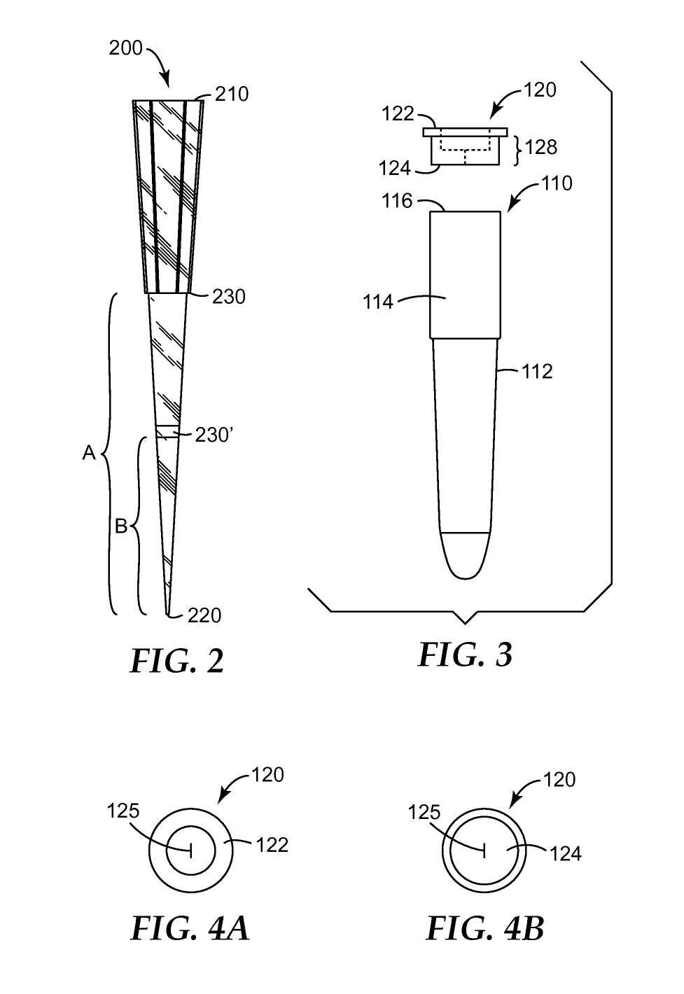

Providing a sample-transfer article comprises providing an article capable of temporarily holding the liquid sample as it is physically moved (i.e., deposited) into the container. Non-limiting examples of sample-transfer articles include pipettes, pipette tips, Pasteur pipettes, transfer pipettes, syringes, droppers, and the like. FIG. 2 shows a side view of one embodiment of a sample-transfer article 200 according to the present disclosure. The sample-transfer article 200 comprises a first end 210 and a second end 220 opposite the first end 210, each end comprising an opening (not shown). A liquid sample (not shown) is drawn into the sample-transfer article 200 through the opening at the second end 220. Optionally, the sample transfer article may further comprise one or more length indexes (230 and 230′, respectively).

A length index (230, 230′) is a visible structure or a mark, on or in the sample-transfer article, which defines a predetermined distance from the opening through which the sample is drawn into the sample conveyor. In FIG. 2, the first length index 230 is a visible edge of a projection; the visible edge located a first predefined distance (“A”) away from the second end 220. The second length index 230′ is a visible filter that is also located a second predefined distance (“B”) away from the second end 220. The one or more length indexes can be used to control the depth to which the sample-transfer device is inserted into the container (e.g., to insure that the second end 220 is inserted into the container to a depth sufficient to contact the sample, to insure the second end is inserted into the container to a depth sufficient to contact a desirable portion of the sample (e.g., a supernatant) while simultaneously avoiding contact with an undesirable portion (e.g., a precipitate) of the sample).

Providing a container, according to the present disclosure, comprises providing a container in which a liquid sample can be processed (e.g., heated, sonicated, mixed with reagents, centrifuged, and/or detected). The container is provided with a pierceable barrier (e.g., a pierceable cap). In some embodiments, the container and the pierceable barrier can be provided separately. In some embodiments, the container and the pierceable barrier can be provided operably coupled to one another.

FIG. 3 shows an exploded side view of one embodiment of a container 110 with a cap 120, according to the present disclosure. The container 110 comprises at least one wall 112 that forms an inner chamber 114. The container 110 further comprises an opening 116 that provides access to the inner chamber 114. Although the illustrated embodiment of the container 110 resembles the shape of a tube that is closed at one end (e.g., a test tube or a microassay tube), it is contemplated that the container 110 could be configured in other shapes (e.g., similar to a flask, a microwell, and the like). The container 110 can be fabricated from a variety of materials such as, for example glass, metal, plastic, provided the material is compatible with the heating procedure and, if applicable, an analyte detection procedure.

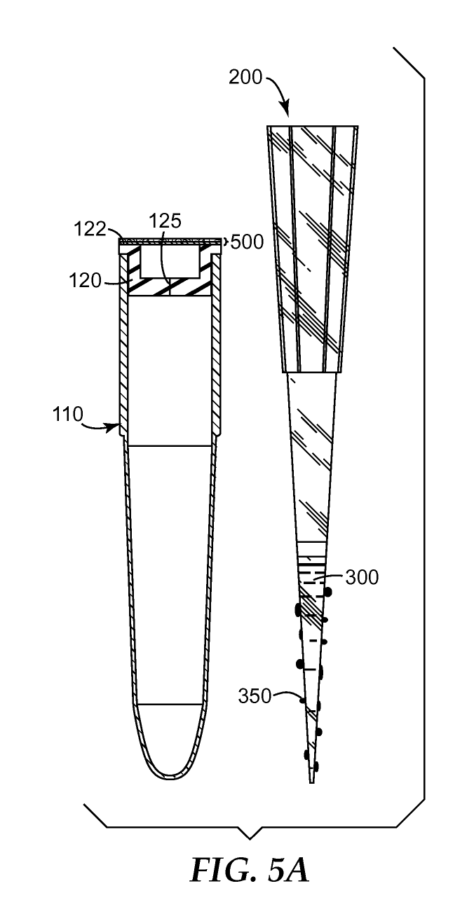

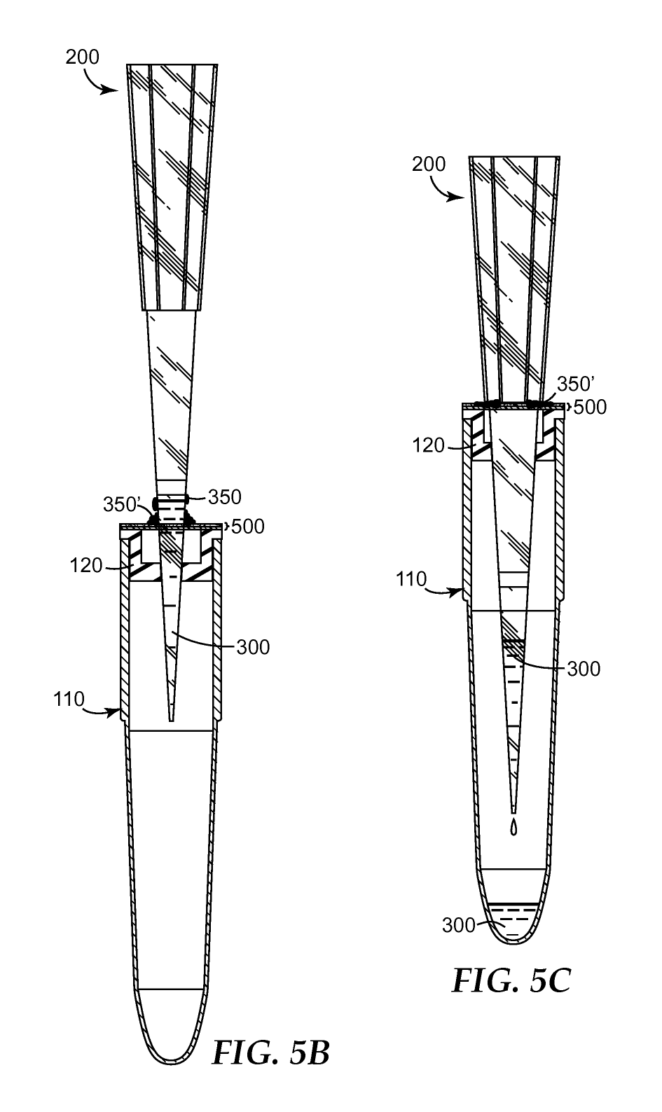

Referring back to FIG. 1, the method of the present disclosure comprises the step 92 of operably coupling the cap and the container. As shown in FIG. 3, the cap 120 is dimensioned to form a closure covering the entire area of the opening 116. Thus, in some embodiments, the cap 120 is shaped and proportioned to seal the opening 116 of the container. The cap includes an outer surface 122 and an inner surface 124. The cap 120 further can include a projected portion 128, at least part of which can be inserted into the opening 116 of the container 110. In some embodiments, the cap 120 can be affixed by press-fitting or friction-fitting a portion (e.g. a projected portion) of the cap 120 into the opening 116 of the container 110. Additionally or alternatively, the cap can be affixed to the container using an adhesive to form a seal between the cap and the opening and/or the wall of the container (not shown). In some embodiments, the cap 120 is affixed to the container 110 before the sample is deposited into the container. In these embodiments, the sample can be deposited into the container by inserting a sample-transfer article (e.g., a pipette, a pipette tip) through the slit in the cap and releasing the sample into the inner chamber of the container, as shown in FIGS. 5A-C. In some embodiments (not shown), the sample is deposited into the container before the cap is affixed to the container.

FIGS. 4A and 4B show plan views of the outer surface 122 and inner surface 124, respectively, of the cap 120 of FIG. 3. The cap 120 includes a pierceable region (e.g., a thinner region or a slit 125, which extends through the cap 120 from the outer surface 122 to the inner surface 124). A non-limiting example of a suitable cap 120 with a pierceable region is the split-cap TPE plug style cap available from Micronic North America, LLC (McMurray, Pa.).

Optionally, the cap can be provided as a two-dimensional mat comprising a plurality of caps (e.g., the Pierceable TPE Capmat, available from Micronic North America, not shown). Alternatively, the cap can be provided as a one-dimensional strip (e.g., the Pierceable TPE Capband-8 or the TPE Capband 12, available from Micronic North America). Preferably, in the mat or the strip, the spacing between the caps matches the spacing typically used to separate containers when they are in use in use (e.g., the typical spacing used in a 96-well plate or heating block).

The method of the present disclosure further comprises providing a pierceable layer. In some embodiments, the pierceable layer comprises a thin (e.g., less than or equal to about 1.25 mm thick) film (e.g., a polymer film) or nonwoven material. Preferably, the pierceable layer is fabricated from a material (e.g. an elastic material) that, after being pierced by an object (e.g., a pipette tip), the layer substantially conforms to the perimeter of the object as the object penetrates the layer. By conforming to the perimeter of the object as the object penetrates the layer, the layer effectively wipes liquid and/or solid residues off the object as it passes through the layer. Even more preferably, the pierceable layer comprises a porous material (e.g., a perforated polymer film such as the perforated polyethylene film used in 3M TRANSPORE Medical Tapes or porous, paper-based 3M MICROPORE Medical Tapes, both available from 3M Company, St. Paul, Minn.; open-cell foam such as BASOTECT melamine foam available from BASF, Florham Park, N.J.). Advantageously, the use of a porous pierceable layer instead of a non-porous pierceable material may reduce the possibility of back-pressure in the container when the sample is ejected from the sample-transfer article into the container.

In some embodiments, the pierceable layer may comprise an absorbent material (e.g., a water-absorbent nonwoven material, a rayon or cellulosic polymer). In some embodiments, the pierceable layer may comprise two distinct materials. For example, the layer may comprise a substantially nonabsorbent film and an absorbent nonwoven that optionally are coupled together (e.g., via an adhesive). The two materials can be configured with the nonabsorbent film facing the cap or with the absorbent material facing the cap.

Referring back to FIG. 1, the method further comprises the step 93 of disposing the pierceable layer adjacent a cap that is operably coupled to a container. In use, the layer is disposed adjacent a pierceable region of a cap such that, in one motion, an object can pass through the layer and the pierceable region of the cap and into the container. In any embodiment, the layer can be detachably coupled (e.g., via an adhesive tape, a clamp) to the container. In any embodiment, the layer can be detachably coupled to the cap (e.g., via an optional adhesive layer disposed on at least a portion of one major surface of the layer), as described herein below.

The method further comprises the step 94 of urging the sample-transfer article through the layer and the cap. FIGS. 5A-C include a series of drawings that illustrate the cleansing function of a pierceable layer according to the present disclosure. FIG. 5A shows a side view of one embodiment of a container 110 (as shown in FIG. 3) operably coupled with a pierceable cap 120 (as shown in FIGS. 4A-B). Disposed adjacent the upper surface 122 of the cap 120 is a pierceable layer 500. Positioned proximate the pierceable region (i.e., slit 125) of the cap 120 is a sample-transfer article 200 as described herein. The interior of the sample-transfer article 200 holds a liquid sample 300 and the exterior surface of the sample-transfer article 200 includes a plurality of residue droplets 350 adhered to thereto.

FIG. 5B shows a side view of the sample-transfer article 200 of FIG. 5A after it is inserted through the pierceable layer 500 and cap 120 of FIG. 5A. Contact between the sample-transfer article 200 and the nonabsorbent film 505 and/or absorbent nonwoven 510 substantially removes the residue droplets 350 from the surface of the sample transfer article 200 as it passes through the pierceable layer 500. In some embodiments (not shown), the nonabsorbent film further can comprise a perforation or a plurality of perforations.

A method according to the present disclosure further comprises the step 95 of transferring a portion of the sample into the container. Typically, the sample comprises a liquid and/or is suspended in a liquid. The liquid-containing sample can be deposited into the container using a sample-transfer article such as a pipette, for example. FIG. 5C shows a side view of the sample transfer article 200, container 110, cap 120, and pierceable layer 500 of FIG. 5B as the sample transfer article 200 is delivering the liquid sample 300 into the container 110. The residue droplets 350 are absorbed by the absorbent nonwoven 510 and, thus, are not deposited onto the outer surface of the cap 120. Advantageously, this prevents the deposition or accumulation of extraneous sample material on the outer surface of the cap 120, where it may be transferred unintentionally and/or unknowingly to a sample-transfer article that is subsequently inserted through the cap.

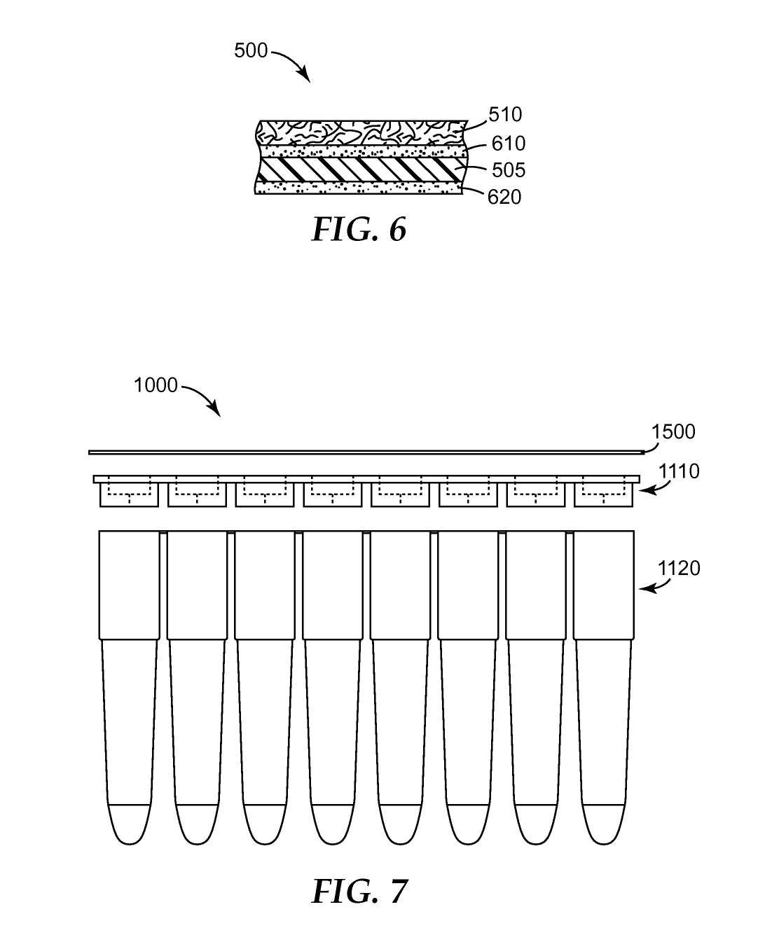

FIG. 6 shows a cross-sectional view of the pierceable layer of FIG. 5A. In the illustrated embodiment, the pierceable layer 500 includes a nonabsorbent film 505 proximate the cap 120 and an absorbent nonwoven material 510 disposed thereon. The nonabsorbent film 505 is coupled to the absorbent nonwoven material 510 via an adhesive layer 610. The nonabsorbent film 505 is coupled to the outer surface of the cap (not shown in FIG. 6) via adhesive layer 620.

The method further comprises the step 96 of separating the pierceable layer for the outer surface of the cap. Separating the pierceable layer may comprise detaching the layer from the container and or, the cap (e.g., detaching a clamp, decoupling an adhesive bond that secures the layer to the container, the cap, and/or the outer surface of the cap). After the pierceable layer is separated from the outer surface of the cap, the layer conveniently can be discarded and the sample can further be processed. Further processing may include, for example, chemical processes (e.g., extraction and/or purification), physical processes (e.g., freezing), storage processes, and/or detection processes.

In any of the above embodiments, providing a container may further comprise providing a container with a reagent disposed therein. The reagent may facilitate cell lysis. In some embodiments, the reagent may be dissolved and/or suspended in an aqueous liquid. In some embodiments, the reagent may be substantially water-free (e.g., a dry powder or a dried-down liquid coating).

In any embodiment, providing a container may further comprise providing a container with a liquid disposed therein. In some embodiments, the liquid may comprise an aqueous suspending medium and/or diluent such as, for example, water, a saline solution, or an aqueous buffer solution. In some embodiments, a reagent may be dissolved and/or suspended in the liquid.

In some embodiments, it may be advantageous to process simultaneously a plurality of samples using the method of the present disclosure. In these embodiments, a plurality of containers (e.g., microtubes) can be placed in an appropriate holder (e.g., tube rack) such that the spacing is suitable for the use of a multichannel pipettor (e.g. a Rainin PIPET-LITE LTS 12-channel (20 μl to 200 μl) multichannel pipette (Mettler Toledo, Columbus, Ohio). Individual caps may be affixed to each of the plurality of containers or a strip or a mat comprising a plurality of caps may be used to affix a cap to each of the plurality of containers either before or after depositing a sample into each of the plurality of containers.

FIG. 7 shows an exploded side view of one embodiment of an assembly 1000 for processing a plurality of samples. Optionally, the samples can be processed simultaneously using a multichannel sample-transfer device (e.g., a multichannel pipettor). The assembly 1000 comprises a container strip 1110 with a corresponding cap strip 1120 and a unitary pierceable layer strip 1500. A multichannel pipettor can be used to deposit simultaneously, optionally through the slits in each cap, a sample into each of the plurality of containers.

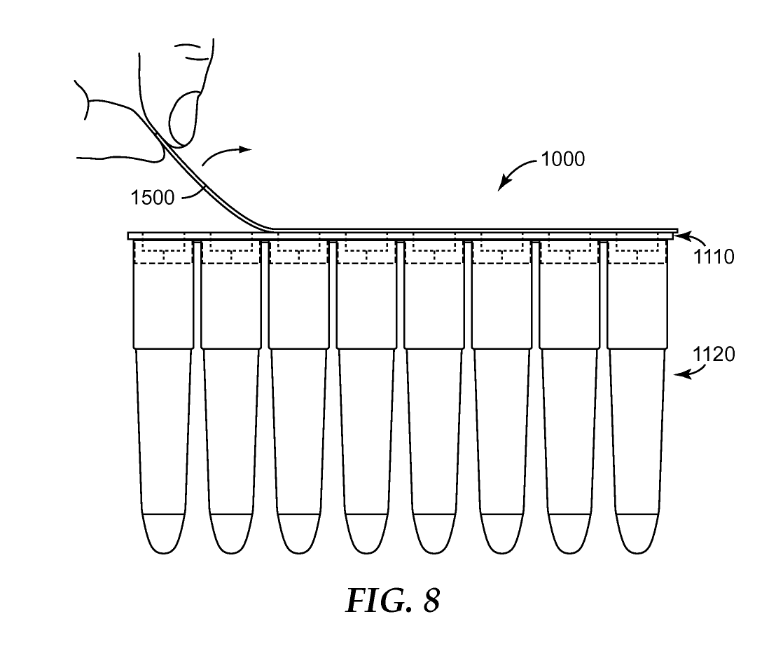

Thus, the present disclosure provides a method of processing a plurality of samples for analysis. The method can comprise providing a plurality of samples or a plurality of aliquots of a single sample; a plurality of containers, optionally interconnected in a one-dimensional strip or two-dimensional mat of containers; a plurality of caps, optionally in a one-dimensional strip or two-dimensional mat of caps; and a plurality of pierceable layers or a unitary pierceable layer strip. Each of the plurality of containers can comprise at least one wall that forms an opening and an inner chamber. Each of the plurality of caps can include an outer surface, an inner surface, and an elastically-deformable slit. The method further can comprise affixing the plurality of caps to the plurality of containers. The method further can comprise disposing the pierceable layer adjacent a plurality of caps. The method further can comprise depositing at least a portion of the plurality of samples or sample aliquots, optionally through a plurality of pierceable caps, into the plurality of containers. The method further can comprise separating the pierceable layer or plurality of pierceable layers from a plurality of caps. Advantageously, when the pierceable layer is provided as a unitary layer strip 1500, the layer strip can be separated from a plurality of capped containers (container strip 1110 and cap strip 1120), as shown in FIG. 8. In any of the embodiments, the pierceable layer strip can be coupled to one or more or a plurality of containers or one or more of a plurality of caps (e.g., the outer surface of the one or more caps), as described above. Optionally, the pierceable layer can be detachably coupled to one or more containers or one or more caps.

In any of the above embodiments, the method further can comprise providing a second sample transfer article having a second sample delivery end and urging the second sample delivery end through the cap (e.g., to remove all or a portion of the sample from the container after it has been stored in the container and/or has been subjected to a physical or chemical process while in the container). In some embodiments, urging the second sample delivery end through the cap further can comprise urging the second sample delivery end through a slit, if present, in the cap.

The present disclosure also provides kits. The kit may be used to prepare a sample or a plurality of samples for analysis.

In one aspect, a kit can comprise at least one container configured to prepare a sample for analysis, a cap, a container, a pierceable layer, and a means for attaching the pierceable layer to the cap and/or to the container. The container has a wall that forms an opening and an inner chamber. The cap is shaped and proportioned to couple with the container and seal the opening. The cap includes an inner surface, an outer surface and a pierceable region to permit the passage of the sample delivery end through the cap. In some embodiments, the pierceable region further can comprise a slit extending from the outer surface to the inner surface of the cap. The means for attaching the pierceable layer to the cap or the container include, for example, an adhesive, a fastener (e.g., a hook-and-loop fastener), and a clamp. In some embodiments, the pierceable layer can be provided in the kit with the means for attachment already coupled there to (e.g., the pierceable layer can be provided with an adhesive layer coated thereon).

In any embodiment of the kit, the pierceable layer may comprise one or more materials (e.g., in layers that are optionally coupled to each other, for example, via an adhesive). The materials may include a porous polymeric film and/or a nonwoven material, as described herein. In any embodiment, the nonwoven material may comprise a water-absorbent nonwoven material.

In any embodiment, the kit further can comprise a sample-transfer article. The sample-transfer article can comprise a pipette, a pipette tip, a Pasteur pipette, or a syringe.

In any embodiment of the kit, the cap further can comprise a plurality of connected, spaced-apart caps, wherein each spaced-apart cap is shaped and proportioned to seal the opening of one of the plurality of containers, as described herein. In any embodiment, the kit further can comprise a liquid disposed in the inner chamber of the container. In any embodiment, the kit further can comprise a detection reagent or a detection device, as described herein. In any embodiment, the kit can further comprise instructions for conducting any embodiment of the method according to the present disclosure.

### Embodiments

Embodiment A is a method of processing a sample for analysis, comprising:

providing:
a first sample transfer article having a first sample delivery end and a sample reservoir having a sample disposed therein;
a container with an opening and an inner chamber;
a cap that is shaped and proportioned to couple with the container and seal the opening; the cap having an inner surface and an outer surface with a pierceable region;
   wherein the pierceable region permits the passage of the sample delivery end through the cap and into the inner chamber; and
a pierceable layer;

operably coupling the cap with the container;

disposing the layer adjacent a portion of the outer surface;

urging the first sample delivery end through the layer and the cap;

transferring a portion of the sample into the container; and

separating the layer from the outer surface.

Embodiment B is the method of embodiment A, wherein providing a cap further comprises providing a cap that includes an elastically-deformable slit extending from the outer surface to the inner surface, wherein urging the first sample delivery end through the cap further comprises urging the first sample delivery end through the cap via the slit.

Embodiment C is the method of embodiment A or embodiment B, further comprising:

providing a second sample transfer article having a second sample delivery end; and

urging the second sample delivery end through the cap.

Embodiment D is the method of embodiment C, wherein urging the second sample delivery end through the cap further comprises urging the second sample delivery end through the slit.

Embodiment E is the method of any one of the preceding embodiments, wherein providing a container and a cap further comprises providing the container with the cap operably coupled there to.

Embodiment F is the method of any one of the preceding embodiments, wherein providing a cap and a pierceable layer further comprises providing the cap with the layer coupled to the outer surface.

Embodiment G is the method of any one of embodiments A through D, wherein disposing the layer adjacent the outer surface further comprises coupling the layer to the outer surface.

Embodiment H is the method of embodiment G, wherein coupling the layer to the region or outer surface further comprises detachably coupling the layer to the outer surface.

Embodiment I is the method of any one of the preceding embodiments, wherein the pierceable layer comprises a water-absorbent nonwoven material, wherein urging the sample delivery end through the layer further comprises absorbing into the layer at least a portion of sample adhered to the outside of the sample transfer article.

Embodiment J is an article, comprising:

a container with an opening and an inner chamber;

a cap that is shaped and proportioned to couple with the container and seal the opening; the cap having an inner surface, an outer surface, and a pierceable region; and

a pierceable layer;

wherein the cap is operably coupled with the container and the layer is coupled to a portion of the outer surface.

Embodiment K is the article of embodiment J, wherein the cap further comprises a pierceable region to permit the passage of a sample delivery end of a sample transfer article through the cap and into the inner chamber.

Embodiment L is the article of embodiment K, wherein the pierceable region further comprises an elastically-deformable slit extending from the outer surface to the inner surface.

Embodiment M is the article of any one of embodiments J through L, further comprising a cell lysis agent disposed in the inner chamber.

Embodiment N is the article of any one of embodiments J through M, further comprising an aqueous liquid disposed in the inner chamber.

Embodiment O is the article of any one of embodiments J through N, wherein the layer is detachably coupled to the outer surface.

Embodiment P is a kit, comprising:

a container with an opening and an inner chamber;

a cap that is shaped and proportioned to couple with the container and seal the opening; the cap having an inner surface, an outer surface, and a pierceable region to permit the passage of the sample delivery end through the cap and into the inner chamber;

a pierceable layer; and

means for coupling the pierceable layer to the cap and/or to the container.

Embodiment Q is the kit of embodiment P, wherein the pierceable layer comprises a film or a nonwoven material.

Embodiment R is the kit of embodiment P or embodiment Q, wherein the pierceable layer comprises a water-absorbent nonwoven material.

Embodiment S is the kit of any one of embodiments P through R, wherein the pierceable layer comprises a perforated polyethylene film and/or a porous paper tape.

Embodiment T is the kit of any one of embodiments P through S, further comprising a sample transfer article.

Embodiment U is the kit of any one of embodiments P through T, wherein providing a cap further comprises providing an article comprising a plurality of connected, spaced-apart caps, wherein each spaced-apart cap is shaped and proportioned to seal the opening of one of a plurality of containers.

Embodiment V is the kit of any one of embodiments P through U, wherein the at least one container further comprises a liquid disposed in the inner chamber.

Embodiment W is the kit of any one of embodiments P through V, further comprising a detection reagent or a detection device.

The present invention is illustrated by the following examples. It is to be understood that the particular examples, materials, amounts, and procedures are to be interpreted broadly in accordance with the scope and spirit of the invention as set forth herein.

### EXAMPLES

Materials
Phenol Red Dye, part number P3532, Sigma Chemical Co. (St. Louis, Mo.) (3.5 mg/mL in 50 mM Tris buffer, pH 8.0)
TPE Plug Style Caps, Capband-8, Micronic North America, McMurray, Pa.
1.1 mL minitube, part number MTS-8-C-R, Axygen, Inc. Union City, Calif.
3M TRANSPORE Medical Tape (Part No. 1527-3, 3M Company, St. Paul, Minn.)
3M MICROPORE Medical Tape (Part No. 1530-3, 3M Company, St. Paul, Minn.)

### Example 1

### Preparation of a Pierceable Layer Strip

The 3M TRANSPORE tape and 3M MICROPORE tape were cut into strips approximately 76 mm long and approximately 9.5 mm wide. The adhesive side of the 3M MICROPORE tape was pressed against the non-adhesive side of the 3M TRANSPORE tape such that the strip of 3M MICROPORE tape substantially superimposed the strip of 3M TRANSPORE tape. In this configuration, the adhesive side of the 3M TRANSPORE tape formed one major surface (i.e., the “lower surface”) of the pierceable layer strip and the non-adhesive side of the 3M MICROPORE tape formed the other major surface (i.e., the “upper surface”) of the pierceable layer strip.

Use of a Pierceable Layer to Prevent Transfer of a Liquid Sample to the Surface of a Microtube Cap.

Three strips of minitubes (8 tubes/strip) designated “A”, “B”, and “C”, respectively, were placed into a tube rack. Each strip of tubes was firmly capped with a strip of eight TPE plug-style caps with the “outer surface” of the caps facing away from the tubes. The lower surface of the pierceable layer strip of EXAMPLE 1 was manually pressed against the outer surface of the caps of minitube strip “A” to form an assembly as depicted in FIG. 7.

A 20-μL pipettor was used to transfer an aliquot (20 microliters) of the Phenol Red solution into each of the tubes in minitube strips “A” and “B”. Minitube strip “C” was a control that did not receive any Phenol Red solution. The pipette tip was inserted through the pierceable layer (when present), through the slit in the cap, and into the tube, where the phenol red solution was deposited into the tube.

The pierceable strip was peeled off minitube strip “A”, as shown in FIG. 7. Forty microliters of distilled water was pipetted into the pierceable region (depression with slit) on the upper surface of each cap in minitube strips “A”, “B”, and “C”. (The depression on the upper surface of the cap can be seen in FIGS. 3 and 4A.) After lavaging the pierceable region with the forty microliters of water, the water was transferred to a PCR tube (Biotix NEPTUNE part no. 3426.8AS, Biotix (San Diego, Calif.) and placed into a fluorometer (Photal FLUODIA T70 fluorometer, Otsuka Electronics, Fort Collins, Colo.). The samples were irradiated with 488 nm light and fluorescent emission was measured at 620 nm. The results are shown in Table 1. The results indicate that the pierceable strip prevents the transfer of about >98% of the phenol red residue that would otherwise transfer to the cap when transferring a sample into the tube through the cap.

TABLE 1
Fluorescent material collected from the caps of minitubes.
All results are reported in relative light units. Each result
is the average of the eight tubes in the strip.

| | Average RLU | Std. Dev. |
|---|---|---|
| Minitube Strip | (×1000) | (×1000) |
| A (with pierceable strip) | 468 | 81 |
| B (no pierceable strip) | 1,435 | 406 |
| C (control - no phenol red) | 449 | 74 |

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A method of processing a sample for analysis, comprising:
providing:
first and second sample transfer articles, each sample transfer article having a first sample delivery end and a sample reservoir having a sample disposed therein;
a container with an opening and an inner chamber;
a cap that is shaped and proportioned to couple with the container and seal the opening; the cap having an inner surface and an outer surface with a pierceable region;
wherein the pierceable region permits the passage of the first sample delivery end through the cap and into the inner chamber; and
a pierceable layer;
operably coupling the cap with the container;
disposing the layer adjacent a portion of the outer surface;
urging the first sample delivery end through the layer and the cap;
transferring a portion of the sample into the container; and
separating the layer from the outer surface;
after separating the layer from the outer surface, urging the sample delivery end of the second sample transfer article through the cap.

2. The method of claim 1, wherein providing a cap further comprises providing a cap that includes an elastically-deformable slit extending from the outer surface to the inner surface, wherein urging the first sample delivery end through the cap further comprises urging the first sample delivery end through the cap via the slit.

3. (canceled)

4. The method of claim 1, wherein providing a container and a cap further comprises providing the container with the cap operably coupled there to.

5. The method of claim 1, wherein providing a cap and a pierceable layer further comprises providing the cap with the layer coupled to the outer surface.

6. The method of claim 1, wherein disposing the layer adjacent the outer surface further comprises coupling the layer to the outer surface.

7. The method of claim 1, wherein the pierceable layer comprises a water-absorbent nonwoven material, wherein urging the sample delivery end through the layer further comprises absorbing into the layer at least a portion of sample adhered to the outside of the sample transfer article.

8. An article, comprising:
a container with an opening and an inner chamber;
a cap that is shaped and proportioned to couple with the container and seal the opening; the cap having an inner surface, an outer surface, and a pierceable region; and
a pierceable layer comprising a nonwoven material;
wherein the cap is operably coupled with the container and the layer is coupled to a portion of the outer surface.

9. The article of claim 8, wherein the cap further comprises a pierceable region to permit the passage of a sample delivery end of a sample transfer article through the cap and into the inner chamber.

10. The article of claim 9, wherein the pierceable region further comprises an elastically-deformable slit extending from the outer surface to the inner surface.

11. The article of claim 8, further comprising a cell lysis agent disposed in the inner chamber.

12. The article of claim 8, further comprising an aqueous liquid disposed in the inner chamber.

13. The article of claim 8, wherein the layer is detachably coupled to the outer surface.

14. A kit, comprising:
a container with an opening and an inner chamber;
a cap that is shaped and proportioned to couple with the container and seal the opening;
the cap having an inner surface, an outer surface, and a pierceable region to permit the passage of the sample delivery end through the cap and into the inner chamber;
a pierceable layer; and
means for coupling the pierceable layer to the cap and/or to the container.

15. The kit of claim 14, wherein the pierceable layer comprises a film or a nonwoven material.

16. The kit of claim 14, wherein the pierceable layer comprises a water-absorbent nonwoven material.

17. The kit of claim 14, wherein the pierceable layer comprises a perforated polyethylene film and/or a porous paper tape.

18. The kit of claim 14, wherein providing a cap further comprises providing an article comprising a plurality of connected, spaced-apart caps, wherein each spaced-apart cap is shaped and proportioned to seal the opening of one of a plurality of containers.

19. The kit of claim 14, wherein the at least one container further comprises a liquid disposed in the inner chamber.
